(19) United States
(12) Patent Application Publication
(10) Pub. No.: US 2017/0188978 A1
(43) Pub. Date: Jul. 6, 2017

(54) SYSTEM AND METHOD OF MEASURING HEMODYNAMIC PARAMETERS FROM THE HEART VALVE SIGNAL

(71) Applicant: Aventusoft, LLC, Boca Raton, FL (US)

(72) Inventors: Kaustubh Kale, Royal Palm Beach, FL (US); Mahdi Esfahanian, Boca Raton, FL (US); Diego Pava, Delray beach, FL (US); Luis Gonzalo Sanchez Gilraldo, Miami, FL (US)

(73) Assignee: Aventusoft, LLC, Boca Raton, FL (US)

(21) Appl. No.: 15/397,037

(22) Filed: Jan. 3, 2017

Related U.S. Application Data
(60) Provisional application No. 62/274,761, filed on Jan. 4, 2016.
(60) Provisional application No. 62/274,770, filed on Jan. 4, 2016.

(51) Int. Cl. A61B 5/00; A61B 5/029; A61B 5/02; A61B 5/0245; A61B 5/0402; A61B 5/021

(52) U.S. Cl. CPC A61B 5/7278; A61B 5/0402; A61B 5/021; A61B 5/02028; A61B 5/0245; A61B 5/029; A61B 5/6823; A61B 2562/0219

(57) ABSTRACT

A system and method for monitoring and diagnosing heart conditions may include capturing and processing a composite heart signal and isolating individual components of such a composite signal. The disclosed techniques of measuring hemodynamic parameters may extract information contained in cardio-pulmonic vibrations. In operation, a system and method may separate different vibration signals along with event time information with respect to a synchronous electrocardiogram signal, and may provide for measurement of hemodynamic parameters from these separated signals.

This application claims the benefit of U.S. provisional patent application Ser. No. 62/274,770, filed Jan. 4, 2016, and U.S. provisional patent application No. 62/274,761, filed Jan. 4, 2016; the subject matter of both of these applications is incorporated herein by reference in its entirety. The entire disclosure of U.S. Pat. No. 8,475,396, entitled ‘Method and system of an acoustic scene analyzer for body sounds’ is also incorporated herein by reference.

The subject matter described in this application was developed with government support under Government Grant No. 1456401, awarded by the National Science Foundation. The subject matter described in this application was developed with government support under Government Grant No. R43MD009556, awarded by the National Institutes of Health. The U.S. Government may have certain rights in the invention.

### FIELD OF THE DISCLOSURE

Aspects of the disclosed subject matter relate generally to cardiac health monitoring, and more particularly to a system and method of measuring hemodynamic parameters from captured cardiac signals along with an electrocardiogram signal for the measurement of heart functions.

### BACKGROUND

Heart disease is the leading cause of death accounting for more than one-third (33.6%) of all deaths in the United States. Overall cardiac health can be significantly improved by proper triage. Minimally invasive and non-invasive ultrasound techniques (e.g., echocardiogram, or EKG or ECG) are standard procedures, but the requirements of expensive devices and skilled operators generally limit their applicability. The following are some of the various types of heart diseases and related disorders that may be diagnosed and treated using hemodynamic parameters measured from separated heart signals: coronary artery disease; heart murmurs and valve abnormalities; heart failure; congestive heart failure; heart rhythm abnormalities (arrhythmias); vascular disease; congenital heart disease; cardiac resynchronization; and risk factor modification. A physician may work with patients to perform a comprehensive evaluation and to design a personalized plan of care aimed at keeping each patient healthy.

The cardio-pulmonary system, which comprises respiratory components, snoring components, and cardiac components, creates vibrations during each cardiac cycle. These vibrations are generally related to lung sounds (due to, for instance, mechanical expansion and contraction, air inhalation, and air exhalation) and heart sounds (due to mechanical operations and muscle contractions, for example), as well as measurable sounds caused by acceleration and deceleration of blood due to abrupt mechanical opening and closing of the valves during the cardiac cycle. The opening and closing time of these valves with respect to the EKG signal are generally referred to as cardiac time intervals.

Cardiac time intervals are related to cardiac physiology, mechanics, and hemodynamics. As left ventricular (LV) systolic function deteriorates causing changes in the hemodynamics, the time it takes for myocardial myocytes to achieve an LV pressure equal to that of the aorta increases, resulting in a prolongation of isovolumic contraction time, which in turn causes deviation of the cardiac time intervals from the normal range. Furthermore, the ability of myocardial myocytes to maintain the LV pressure decreases, resulting in reduction in the ejection time (ET). Therefore, the cardiac time intervals contain information on hemodynamics, and both systolic and diastolic performance, which may be useful to identify subtle impairments in the cardiac function in patients at risk of future cardiovascular disease. Conventional methodologies do not isolate such sounds caused by or emanating from the respective components in the cardio-pulmonary system. Therefore, there is a need for an improved system and method of capturing multi-channel vibration signals along with an electrocardiogram signal for measuring hemodynamic parameters.

### SUMMARY OF THE DISCLOSURE

The following presents a simplified summary of the disclosure in order to provide a basic understanding of some aspects of various embodiments disclosed herein. This summary is not an extensive overview of the disclosure. It is intended neither to identify key or critical elements of the disclosed embodiments nor to delineate the scope of those embodiments. Its sole purpose is to present some concepts of the invention in a simplified form as a prelude to the more detailed description that is presented later.

The present disclosure describes a system and method of measuring hemodynamic parameters from separated, identified, and marked heart valve signals. Data may be obtained using a tri-axial accelerometer, for example, or optionally, multiple tri-axial accelerometers placed on different points of a patient's torso. Additionally or alternatively, sonic sensors or microphones may be employed either individually or in combination with accelerometers or other sensor types,

In accordance with one embodiment, a method of measuring hemodynamic parameters is disclosed the method comprising: receiving a composite signal representative of emanations from a plurality of sources associated with a patient's cardio-pulmonary system; isolating an individual signal as a separate component of the composite signal, the individual signal representative of an emanation from one of the plurality of sources; and deriving hemodynamic parameters responsive to the isolating. The method may further comprise providing an output related to the individual signal and the hemodynamic parameters.

In some methods, the receiving comprises employing an array of transducers, wherein each respective transducer in the array is disposed in a respective location on a patient. The isolating may comprise measuring a cardiac time interval for the individual signal with respect to an electrocardiogram signal.

The deriving may comprise utilizing an equation having the form Y=A1+A2*(A3), where Y is the hemodynamic parameter, A1 and A2 denote predetermined numerical constants, and A3 denotes a cardiac time interval or a ratio of two cardiac time intervals. In some implementations, the predetermined numerical constants A1 and A2 are based upon empirical data. Alternatively, the deriving may comprise utilizing a linear regression model equation having the form Y(n)=b+Σwᵢ*xᵢ(n)+ε(n) (where Y(n) is the hemodynamic parameter, xᵢ(n) is the i^{th} cardiac time interval feature from the n^{th} context frame, wᵢ denotes a weight, b denotes a bias parameter, and ε(n) denotes independent and identically distributed zero mean observation noise), or utilizing a non-linear regression model equation having the form Y(n)=f(x(n))+β(n)=Σαⱼ*k(x(n), x(j))+ε(n) (where Y(n) is the hemodynamic parameter, xᵢ(n) is the i^{th} cardiac time interval feature from the n^{th} context frame, αⱼ denotes a weight, k denotes a positive definite kernel, and ε(n) denotes independent and identically distributed zero mean observation noise).

In accordance with another aspect of the disclosed subject matter, a method of determining hemodynamics parameters may generally comprising sensing contractility changes within a heart by identifying a cardiac time interval, sensing an electrocardiogram (EKG) signal representative of heart function, and deriving the hemodynamic measurements using the sensed contractility changes and the EKG signal. In some applications, the sensing contractility changes comprises measuring systolic time intervals of the heart and diastolic time intervals during a period from a start of a Q-wave of the EKG signal to a time when a change is sensed in the cardiac contractility.

The deriving the hemodynamic measurements may generally comprises utilizing contractility estimates to determine a ventricular systolic pressure and a ventricular diastolic pressure. Such a method may further comprise multiplying the diastolic time interval by the ventricular diastolic pressure, multiplying the systolic time interval by the ventricular systolic pressure, and adding the products to obtain a mean pulmonary artery pressure.

Additionally or alternatively, the deriving the hemodynamic measurements may comprise utilizing contractility estimates to determine any one or more of the following: i) a systolic blood pressure and a diastolic blood pressure; ii) an ejection fraction; iii) a stroke volume, wherein a product of the stroke volume and a heart rate determines cardiac output; iv) a vascular resistance; v) a global longitudinal strain; vi) a fluid status of a person; vii) pulmonary artery systolic pressure along with pulmonary artery diastolic pressure; and viii) systolic dysfunction and diastolic dysfunction.

Additionally, a system for monitoring cardiac parameters may generally comprise: a non-invasive cardiac parameter measuring unit for non-invasively measuring a plurality of predetermined non-invasive cardiac parameters from a subject; a conversion unit connected to the non-invasive cardiac parameter measuring unit for converting the non-invasive cardiac parameters into a plurality of invasive cardiac analogues based upon a set of predetermined conversion equations and; a display unit connected to the conversion unit for displaying a vector indicative of a hemodynamic state.

The foregoing and other aspects of various disclosed embodiments will be apparent through examination of the following detailed description thereof in conjunction with the accompanying drawing figures.

### DESCRIPTION OF THE DRAWING FIGURES

FIGS. 1A and 1B are diagrams illustrating components of one system for source separation of cardio-pulmonary signals;

FIG. 2 is a diagram illustrating one alternative for cardio-pulmonary signal capture at a patient's chest;

FIG. 3 illustrates individual streams identified by a system and method of capturing multi-channel vibration signals;

FIG. 4 illustrates a cardiac cycle and representative signals produced by electrocardiogram, acoustic, and accelerometer sensors;

FIG. 5 illustrates one representation of cardio-pulmonary sounds in relation to electrocardiogram signals;

FIG. 6 illustrates one representation of cardiac time intervals estimated from source separated heart valve signals;

FIG. 7 illustrates Ejection Fraction measurements derived from PEP/LVET;

FIG. 8 illustrates blood pressure measurements derived from cardiac time intervals PEP/LVET; and

FIG. 9 illustrates mean pulmonary blood pressure measurement and right ventricular systolic pressure measurement from cardiac time intervals, such as PEP/LVET.

### DETAILED DESCRIPTION

Certain aspects and features of the disclosed subject matter may be further understood with reference to the following description and the appended drawings. In operation, a system and method of measuring hemodynamic parameters may employ cardiac time intervals (described below) calculated from source separated individual cardio-pulmonary signals isolated from composite vibrations captured on the chest wall. Specifically, psychoacoustics may be considered in measuring hemodynamic parameters captured through any of a number of transducers. The disclosed implementations may be operative with respect to various hemodynamic measurements, such as: systolic blood pressure; diastolic blood pressure; pulmonary arterial blood pressure (systolic, diastolic, mean); left ventricular ejection fraction; right ventricular ejection fraction; stroke volume; cardiac output; vascular resistance; global longitudinal strain; fluid status; dP/dTmax; E/E′; pulmonary capillary wedge pressure, Left ventricle end diastolic volume, Systolic Dysfunction Index (SDI); and Diastolic dysfunction index (DDI).

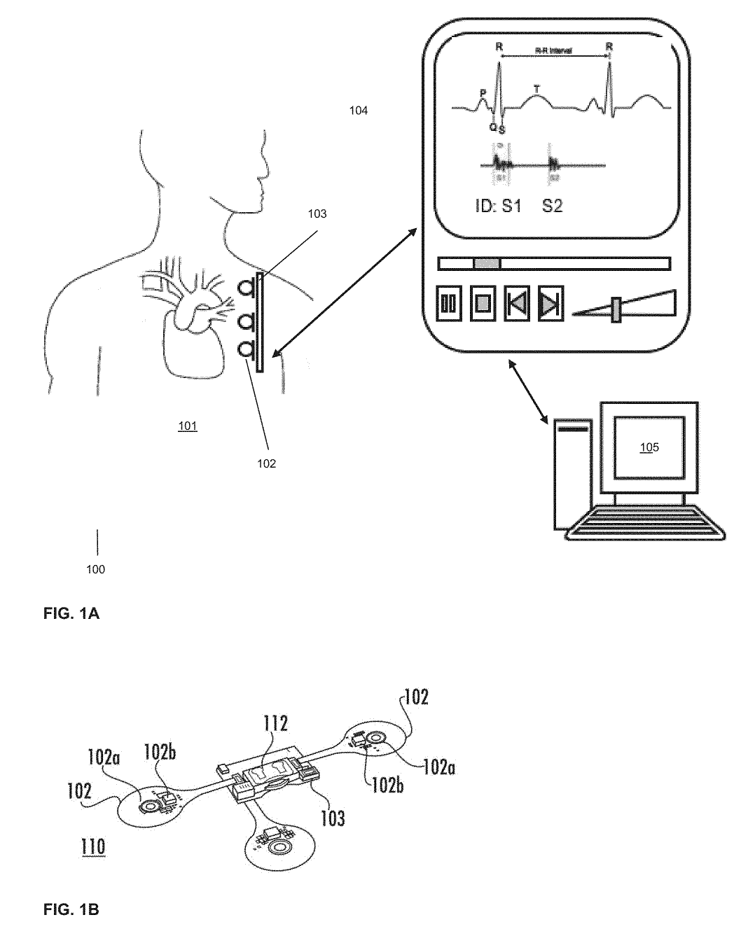

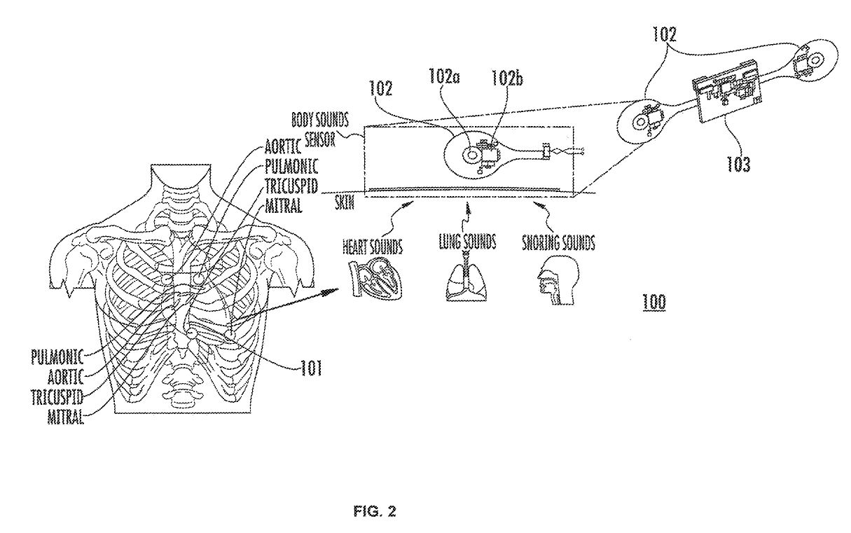

In operation, a system and method of measuring hemodynamic parameters as set forth herein may provide a unique approach for small, portable, robust, fast, and configurable source separation based software with transducer hardware (reference numeral 103 in FIGS. 1A, 1B, and 2). As set forth in detail below, the use of vibration signal pattern analysis and novel psychoacoustics may bypass, mitigate, or overcome issues faced by conventional linear time invariant systems.

As used in the present disclosure, the terms “source separated” and “source separation” refer to an individual or component signal that has been isolated or extracted from a combined or composite “source” signal, and the process of so isolating or extracting such a signal, as the context dictates. In these contexts, an individual signal having been so isolated can be characterized as having been “separated” from the “source” composite signal. Similarly, the term “multi-channel vibration signal” refers to a combined or composite “source” signal representative of vibrations that emanate from a plurality of different cardio-pulmonary components. In this context, each channel of the multi-channel signal may be representative of an individual signal from a respective component in the cardio-pulmonary system. Those of skill in the art will appreciate that aspects of the present disclosure relate specifically to the utility of identifying each individual signal and isolating, extracting, or otherwise acknowledging its individual contribution to the overall composite signal.

Some examples of separated vibration sources include mitral valve opening and closing, aortic valve opening and closing, pulmonary valve opening and closing, tricuspid valve opening and closing, and heart wall motions. A portion of the energy produced by these vibrations lies in the infra-sound range, which falls in the inaudible and low sensitivity range of typical human hearing capabilities, though another portion of the energy produced by these vibrations falls in the audible range for most humans' sensitivities. In some implementations, an accelerometer transducer placed on the chest may capture signals representative of such vibrations and measure energy from both of these ranges. Briefly, heart events are produced by the vibrations of the cardiohemic system, composed of the blood, heart walls, and valves. The vibrations are triggered by the acceleration and deceleration of blood due to abrupt mechanical events of the cardiac cycle. Signals present at the chest wall are the result of the heart muscles, together with the transmission characteristic of the heart and chest wall. A portion of signals produced by these vibrations lies in the human audible frequency range and a portion lies in the lower-frequency inaudible infrasound range. Heart signals recorded on the chest wall are found between 0-1000 Hz with the main energy below 100 Hz. Accordingly, in one embodiment electrocardiogram style pads or sensors may be placed in a non-linear arrangement such that the position and magnitude of an applied force can be localized; wherein the force is generated in response to a body function, for example, a compression or decompression of chest walls responsive to a heart beat, respiratory function or muscle contraction. In another embodiment, these heart signals may be captured using echocardiography images, x-ray images, computed axial tomography images, magnetic resonance images; or data from invasive catheters.

In some embodiments, a source separation analysis technique may be used to extract individual vibration contributions from the composite vibration signal captured on the surface of the chest, or presented through an echocardiography image, x-ray image, computed axial tomography image, or magnetic resonance image. In operation, a system and method may identify or isolate respective contributions of individual vibration signals from the mitral valve, the aortic valve, the tricuspid valve, and the pulmonary valve during individual heart beats. The identified valve signals may be marked, for instance, to indicate a respective start and end of each measured or identified event, and to register each event with respect to the start of an electro-cardiogram (EKG or ECG) signal. The timing of these events relative to the EKG signal—corresponding to the opening and closing of each valve—may be referred to as “cardiac time intervals.”

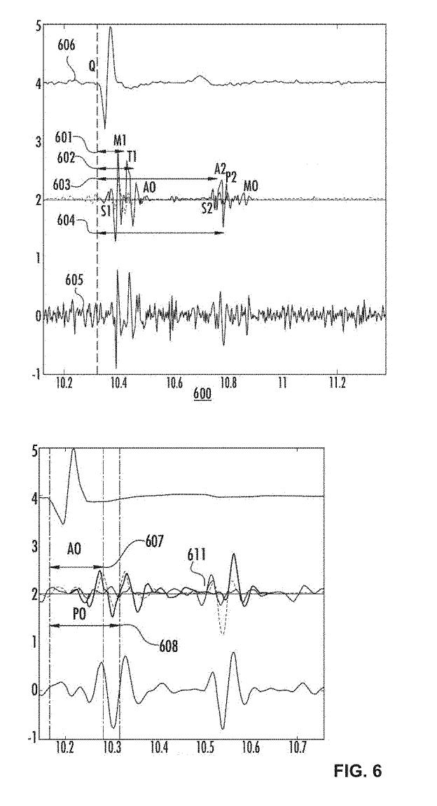

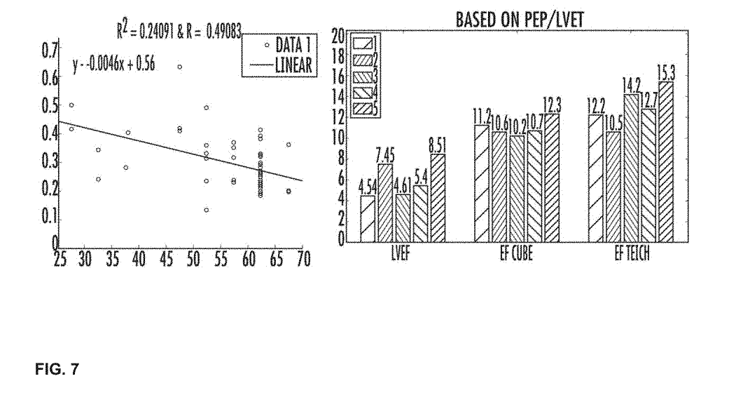

The extracted individual valve vibration objects are aligned (for example, by a microprocessor or other digital processing component) into a signal for each of the four valves. Each signal identifies the source separation of heart valve opening and closing signals for each valve of the composite heart signal. This is shown in FIG. 6, which illustrates a plot showing the source separation of heart valve signals. This alignment of the separated signals is also a unique preparatory step for measuring the hemodynamic parameters as shown in FIG. 7.

In some instances, identification and measurement of cardiac time intervals may be followed by measurement of hemodynamic parameters, which may be derived from, among other factors, the cardiac time intervals. In some implementations, such measurement of hemodynamic parameters may occur substantially simultaneously with identification and measurement of cardiac time intervals. It will be appreciated that various hemodynamic measurements may have utility in different contexts and for different purposes. Some such hemodynamic measurements include systolic blood pressure, diastolic blood pressure, pulmonary arterial blood pressure (systolic, diastolic and mean), left ventricular ejection fraction, right ventricular ejection fraction, stroke volume, cardiac output, vascular resistance, global longitudinal strain, fluid status, dP/dTmax, E/E′, Systolic Dysfunction Index (SDI), and Diastolic dysfunction index (DDI).

As noted above, some of the various types of heart diseases and related disorders that can be diagnosed and treated using the hemodynamic measurements calculated from the separated signals include coronary artery disease, heart murmurs and valve abnormalities, heart failure, congestive heart failure, heart rhythm abnormalities (arrhythmias), vascular disease, congenital heart disease, and risk factor modification. In accordance with the present disclosure, a system and method may measure hemodynamic parameters quickly and accurately without the need for complex equipment or intervention on the part of medical staff or paramedical personnel.

The clinical importance of analyzing cardiac time intervals as a mechanism to provide hemodynamic measurements has been extensively reported in medical literature. Wang et al. (2012) (incorporated herein by reference) evaluated the ability of acoustic cardiography to identify severe systolic dysfunction (left ventricular ejection fraction (LVEF), ±35%) and/or severe diastolic dysfunction (presence of a restrictive LV filling pattern) in patients experiencing heart failure with reduced ejection fraction. The study included 127 adult inpatients, all of whom where afflicted with heart failure and reduced ejection fraction. All patients underwent acoustic cardiography and echocardiography, and the diagnostic accuracy of acoustic cardiography was compared with echocardiography. The authors used a Systolic Dysfunction Index (SDI) to quantify systolic dysfunction on acoustic cardiography. The SDI was based on S3 (third heart sound) score, QRS duration, QR interval, and percent EMAT (Electromechanical activation time), and was mapped onto a scale of 0 to 10, where an SDI greater than 5 indicates an LVEF less than 50%, and an SDI greater than 7.5 indicates an LVEF less than 35% and elevated LV filling pressure. An SDI greater than 5 was the best predictor to discriminate patients with LVEF less than or equal to 35% from those with moderate systolic dysfunction (LVEF between 35% and 50%), associated with an AUC of 0.79 (95% confidence interval (CI), 0.71 to 0.87), with sensitivity of 87% and specificity of 60%. For the subgroup of 122 patients with diastolic dysfunction, an S3 score greater than 4 best predicted a restrictive filling patter, associated with an AUC of 0.76 (95% CI, 0.67 to 0.84), with sensitivity of 81% and specificity of 55%. Toggweiler et al. (2012) (incorporated herein by reference) reported results of a prospective cohort study to evaluate the role of acoustic cardiography in follow-up for left ventricular function among patients who had undergone an anthracycline-containing chemotherapy regimen. The study included 187 patients who had undergone anthracycline treatment for a variety of cancers at a single institution. At baseline, at completion of anthracycline-containing chemotherapy, and at long-term follow up, patients underwent evaluation with echocardiography and acoustic cardiography. Left ventricular function was evaluated with acoustic cardiography using EMAT values. Over a mean follow-up of 3.8 years, the mean LVEF, measured by echocardiography, decreased from 64% at baseline to 61% post chemotherapy (p<0.001) and remained at 61% at late follow up (p<0.01 vs baseline). Mean EMAT increased from 80 ms at baseline to 84 postchemotherapy (p<0.01), and increased to 89 at late follow-up (p<0.01 vs baseline). The relative percent change in EMAT correlated with relative changes in the LVEF (r=−0.33, p<0.01). Eight patients (4%) developed systolic dysfunction. A percent change in EMAT of greater than 12.4% after chemotherapy had a sensitivity and specificity of 88% and 85%, respectively, for identifying patients with systolic dysfunction. The authors recommend the use of acoustic cardiography for monitoring patients based on the high sensitivity and specificity for identifying systolic dysfunction. Chan et al. (2013) (incorporated herein by reference) evaluated the role of acoustic cardiography in the evaluation of the severity of pulmonary arterial hypertension (PAH) in a prospective case-control study of 40 cases with PAH and 130 controls without clinical or hemodynamic evidence of PAH. The intensity (measured by peak-to-peak amplitude and expressed in mV) and complexity (measured as a dimensionless index based on spectral analysis) of S1 and S2 were evaluated. Patients with PAH were found to have significantly greater S2 complexity as compared to those in the control group (p<0.001 for both lead V3 and V4 position); S2 complexity was associated with mean pulmonary artery pressure among patients with PAH (r=0.55, p<0.001).

The two major audible heart sounds in a normal cardiac cycle are the first and second heart sound, S1 and S2, respectively. S1 occurs at the onset of the ventricular contraction during the closure of the mitral and tricuspid-valves. It generally contains a series of low-frequency vibrations, and is usually the longest and loudest heart sound. Audible sub-components of S1 may be associated with the closure of each of the two mitral and tricuspid valves. S2 is generally heard at the end of the ventricular systole, during the closure of the aortic and pulmonic valves. Typically, its frequency is higher than S1, and its duration is shorter. It may also have aortic and pulmonary sub-components. A third, generally low-frequency sound (S3, sometimes referred to as “ventricular gallop”), may be heard at the beginning of the diastole, during the rapid filling of the ventricles. A fourth heart sound (S4, sometimes referred to as “atrial gallop”) may be heard in late diastole during atrial contraction. A fifth low frequency vibration that is below audible frequency range occurs due to the opening of the mitral, tricuspid, aortic and pulmonic valves. Opening snaps of the mitral valve or ejection sound of blood coursing in the aorta may be heard in some cases of valve disease (stenosis, regurgitation). Murmurs are high-frequency, noise-like sounds that may be heard between the two major heart sounds during systole or diastole. They can be innocuous in some cases, but they may also indicate or portend certain cardiovascular defects.

As is generally known, audible S4, possibility indicative of abnormal left atrial filling waves, and audible S3 are commonly noticed in patients with coronary artery disease, left ventricular dysfunction, or both. Extensive correlations of these clinical findings with hemodynamics have been demonstrated, providing the importance of extracting these signals from the composite signals for automated and remote monitoring and diagnosis. As noted above, S3 is generally characterized as a low frequency sound coinciding with the rapid filling phase of ventricular diastole. It is recorded approximately 0.10 to 0.20 seconds after the aortic component of S2, and often corresponds to a rapid filling wave. The major vibrations of S4 usually occur approximately 0.12 to 0.17 seconds after the onset of the P wave of the electrocardiogram. These vibrations usually precede the onset of the QRS complex, except in instances of short P-R intervals. Unless the P-R interval is prolonged, S4 is normally inaudible, although some small, insignificant vibrations may be recorded at low frequency ranges. Significant (i.e., abnormal) vibrations have greater amplitude and pitch, and constitute the audible, and clinically meaningful, S4, which may be readily recorded, even at medium frequency ranges.

Turning now to the drawing figures, FIGS. 1A and 1B are diagrams illustrating components of one system for source separation of cardio-pulmonary signals. In the illustrated arrangement, system 100 is represented as an embedded platform which may be embodied in or comprise a processing platform having digital signal processing capabilities, an application processor, data storage, a display, an input mechanism or interface such as a touch-screen or keypad, microphones, speakers, Bluetooth™ or other near-field communications connectivity, and connection to a network such as the internet via, for example, a wide area network (WAN), wireless fidelity (Wi-Fi) or Ethernet protocols, universal serial bus (USB) hardware, or other networking technologies known in the art or developed in accordance with known principles. It is noted that the present disclosure is not intended to be limited to any particular implementation of the hardware components, network architectures, or communications protocols illustrated and described with reference to FIG. 1.

Reference numeral 101 depicts the auditory environment at a chest of a patient or subject to be monitored. Reference numeral 110 represents a component to be disposed on or near a patient's chest, and includes a transducer array 102 that may be used to capture a composite signal from the environment 101; as noted above, such a composite signal may include one or more signals from the heart, one or more signals from the lungs, one or more signals from the sinus area, trachea, or throat, or a combination of these and other signals. In use, transducer array 102 may be applied to a patient's torso (i.e., chest, abdomen, back, ribcage, etc.) in or proximate to auditory environment 101 from which relevant sound and other vibrations may emanate. In that regard, transducer array 102 may be embodied in or comprise a sonic or an ultrasonic transducer 102a, an accelerometer transducer 102b, or other electromagnetic hardware component such as an EKG sensor or the like. In use, transducer array 102 may be operative to capture or acquire sonic energy, vibrational energy, or both within frequency ranges that are medically or diagnostically relevant as well as to direct or transmit signals representative of such sonic energy or vibrational energy to another component of system 100 for processing or analysis. In the case where transducer hardware is coupled or integrated with a transmitter, a transceiver, or other communications hardware, it may not be necessary to provide an independent or stand-alone component to deliver signals from transducer array 102 to another component of system 100. It will be appreciated that transducer array 102 may be implemented as a single transducer in some implementations where hardware and processing capabilities are suitable for using a single transducer component as opposed to an array of components disposed at different locations relative to the environment 101. Accordingly, the present disclosure is not intended to be limited to any particular implementation of transducer and communications technology associated with transducer array 102; any structural arrangement and hardware implementation that supports the functionality set forth below may be suitable for use at transducer array 102, including those that employ only a single transducer component, those that employ ultrasonic and infrasonic detection technologies, and those that incorporate or integrate near field or other communications capabilities.

As illustrated in FIGS. 1A and 1B, reference numeral 103 represents a wearable microprocessor-based hardware component having, in some instances, digital signal processing capabilities, an application processor, an analog to digital frontend, data storage, an input mechanism such as buttons or a touch-sensitive display, and wireless connectivity to other components of system 100, such as via Bluetooth™, Bluetooth™ low energy, some other near field communication transceiver, Wi-Fi, Ethernet, or USB. The individual hardware elements (reference numeral 112) of wearable component 103 are generally well known in the art, and so details have been omitted from the drawings for clarity. In some implementations, some or all of the functionality described below with reference to wearable component 103 may be executed by a remote processing component 105; in such arrangements, processing power, architectural complexity, and battery or other power requirements at wearable component 103 (other than signal detection and transmission) may be minimized or eliminated.

In one embodiment, wearable component 103 may generally comprise a signal processing module operative (e.g., integrated with hardware 112) to capture or otherwise to receive sensor data from transducer array 102. These data may be synchronized in some instances, for example, with events occurring during a cardiac cycle. In operation, wearable component 103 may be embodied in or comprise a microprocessor, a microcontroller, a digital signal processor (DSP), a field programmable gate array (FPGA), an application specific integrated circuit (ASIC), a programmable logic controller (PLC), or some other electronic hardware component suitable for processing data as set forth herein. Those of skill in the art will appreciate that various hardware architectures may be appropriate at wearable component 103 depending upon, among other things, the processing bandwidth, power requirements, and computational capabilities of other components of system 100, such as remote processing component 105.

In one implementation, suitable hardware 112 resident at or incorporated in wearable component 103 may save or store data (received from transducer array 102) to memory (not shown in FIG. 1) and may communicate some or all of same to other components of system 100. In that regard, reference numeral 104 in FIG. 1A represents a functional block, or “vitals module,” that is operative to calculate or derive vital signs of a patient from the data stream acquired by transducer array 102 and transmitted by wearable component 103; such vital signs may include, but are not limited to, heart rate, breathing rate, EKG signal, skin temperature, and associated data acquired by transducer array 102.

It will be appreciated that, in this context, the term “vitals module” generally refers to a functional block that be embodied in or implemented by a software application resident in memory, for instance, a hardware or firmware component, or both. In the FIG. 1A embodiment, vitals module 104 is illustrated as a discrete component, independent of wearable component 103 and remote processing component 105, though it will be appreciated that some or all of the functionality executed at vitals module 104 may be implemented at these elements, depending upon processing capabilities, memory access, and overall architecture of system 100 and the constituent components 103, 104, and 105, which may operate independently or in cooperation with each other. In the event that vitals module 104 is implemented as a discrete component of system 100 (residing, for example, on a network accessible server or otherwise in “the cloud”), then it may be desirable to encrypt raw sensor data (i.e., those data detected by transducer array 103) or intermediate processed data (i.e., those derived by any processing operation executed by wearable component 103) prior to transmission. Specifically, raw sensor data or intermediate processed data may be encrypted, compressed, or both prior to or in conjunction with transmission to remote processing component 105.

Vitals module 104 may also communicate with a dashboard or other user interface component resident on or operative at remote processing component 105 to facilitate data exchange, login, alerts, notifications, display of processed data, or a combination of these and other functionalities as generally known in the art and described below. As illustrated in FIG. 1A, remote processing component 105 is illustrated as a network-accessible or cloud-based processing component or server that may generally be operative to process streams of data from wearable component 103 for eventual source separation (to the extent that such processing is not performed by operations executed by wearable component 103 itself). During use of system 100, a user may be enabled to see individual streams, composite signals, or both, as well as other information related to or derived from different cardio-pulmonary signals displayed by a suitable hardware. In one embodiment, visualization may be provided by a display associated with wearable component 103; additionally or alternatively, a display may be provided at remote processing component 105. In some instances, it may be desirable to implement vitals module 104 as an independent element of system 100 such that it employs hardware and software sufficient to display data independent of any monitor or display hardware at wearable component 103 or remote processing component 105.

FIG. 2 is a diagram illustrating one alternative for cardio-pulmonary signal capture at a patient's chest. Multiple sensor transducers (such as may be implemented at transducer array 102) that are operable to capture a composite signal at or proximate to an auditory environment 101 are shown. As noted above, each transducer array 102 may acquire raw data comprising or associated with electrocardiogram signals, heart sounds, lung sounds, and snoring sounds; it is appreciated that only a single transducer array 102 may be sufficient for some applications, depending, for instance, upon the nature and operational characteristics of the transducer hardware and the type of raw data sought to be acquired. Reference numeral 103 in FIG. 2 represents the wearable component 103 described above. Wearable component 103 may acquire raw data in the form of signal output from transducer array 102 and, in some embodiments, may provide or enable analog to digital conversation, storage, error correction or other pre-processing, encryption, compression, and transmission of raw data or intermediate processed data to other system components such as vitals module 104, remote processing component 105, or both.

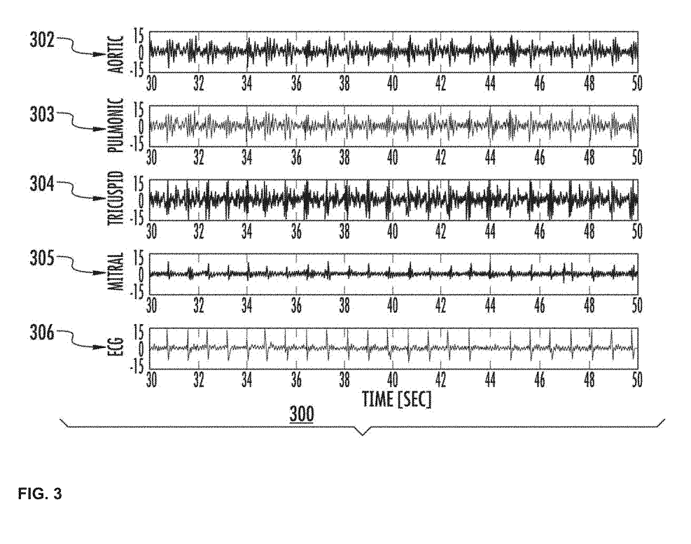

FIG. 3 illustrates individual streams identified by a system and method of capturing multi-channel vibration signals. The various streams are indicated collectively at reference numeral 300. In particular, FIG. 3 illustrates a vibration signal acquired at or proximate to an aortic auscultation location (reference numeral 302), a vibration signal captured at or proximate to a pulmonic auscultation location (reference numeral 303), a vibration signal captured at or proximate to a tricuspid auscultation location (reference numeral 304), a vibration signal captured at or proximate to a mitral auscultation location (reference numeral 305), and an electrocardiogram signal (reference numeral 306).

In another embodiment, the individual streams of data illustrated in FIG .3 may be data from echocardiography images, x-ray images, computed axial tomography images, magnetic resonance imaging apparatus, or invasive catheters or electroencephalogram (EEG), or pulse oximetry devices.

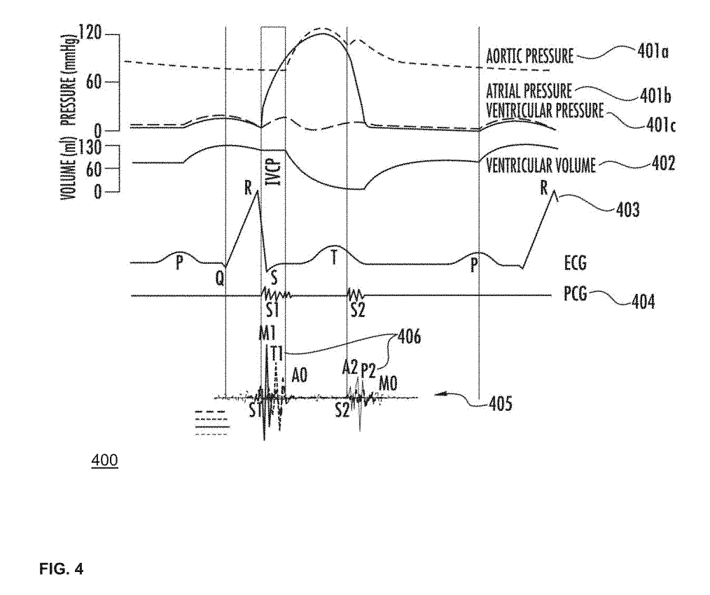

FIG. 4 illustrates a cardiac cycle and representative signals produced by electrocardiogram, acoustic, and accelerometer sensors. In that regard, it will be appreciated that the disclosed system and method draw inspiration from biology with respect to the cardiac cycle as it may relate to and be represented by electrocardiogram signals, accelerometer transducer captured cardiac signals, or both. Reference numeral 400 in FIG. 4 is a representation of a cardiac cycle as interpreted by output of various sensors. Reference numerals 401a-401c in FIG. 4 indicate certain pressure changes during a cardiac cycle, while reference numeral 402 indicates certain volume changes during the same cardiac cycle. Similarly, reference numeral 403 in FIG. 4 indicates electrical changes during a cardiac cycle captured by an electrocardiogram, while reference numeral 404 indicates acoustic changes during a cardiac cycle as captured by an acoustic sensor. Finally, reference numeral 405 in FIG. 4 indicates certain vibration changes during a cardiac cycle as captured by an accelerometer transducer, and reference numeral 406 indicates the different valve opening and closing events as captured by the accelerometer sensor.

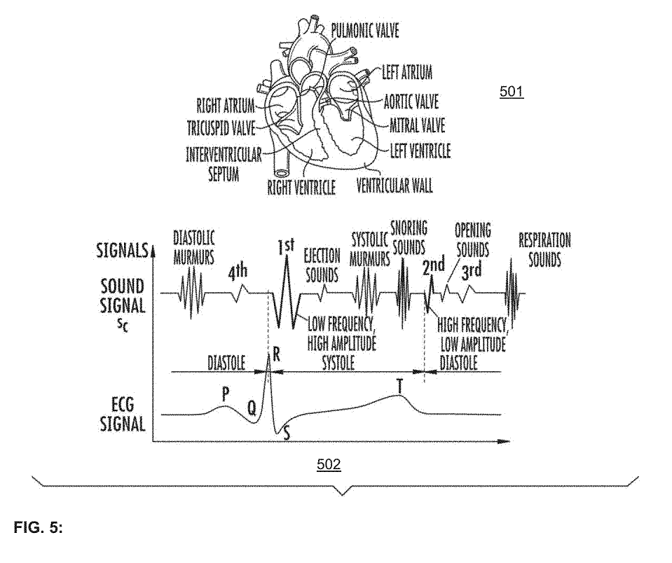

FIG. 5 illustrates one representation of cardio-pulmonary signals in relation to electrocardiogram signals. The top portion of FIG. 5 (at reference numeral 501) provides a representation of the locations from which sounds may emanate during a cardiac cycle. Different sounds (such as those emanating from or caused by the coronary artery, murmurs, S1 sound, S2 sound, S3 sound, S4 sound, ejection sounds, opening sounds, respiratory sounds, breathing, and snoring) that occur during individual heart beats are illustrated in the lower portion of FIG. 5 at reference numeral 502. As indicated, the x-axis in FIG. 5 represents time, and the sounds depicted are registered (i.e., synchronized in time) with respect to cardiac cycle events as represented the electrocardiogram signal. It will be appreciated that reference numeral 501 illustrates the anatomy relevant for the generation of the various sounds.

It will be appreciated that isolating the source of sounds as depicted in FIG. 5 may provide a system and method of monitoring heart function with useful data that are otherwise lost, or at least undifferentiated, in a combined or composite signal. In that regard, a source marking algorithm may allow an improved system and method to mark, isolate, extract, or otherwise differentiate sounds or other signals associated with some or all of the following events: Mitral valve closing (MC); Mitral valve opening (MO); Aortic valve opening (AO); Aortic valve closing (AC); Tricuspid valve closing (TC); Tricuspid valve opening (TO); Pulmonary valve closing (PC); and/or Pulmonary valve opening (PO).

FIG. 6 illustrates one representation of cardiac time intervals estimated from source separated heart valve signals. In an embodiment, extracted individual valve vibration objects may be aligned into a signal for each of the four heart valves across multiple heart beats. FIG. 6 illustrates the source separation of heart valve opening and closing signals 600, wherein: 601 indicates a vibration signal for mitral valve closing, 602 indicates a vibration signal for the tricuspid valve closing, 603 indicates a vibration signal for the aortic valve closing, 604 indicates a vibration signal for the pulmonic valve closing, 605 indicates a composite vibration signal captured by a particular transducer, and 606 indicates an EKG signal captured by the system. Similarly, 607 indicates a vibration signal for the aortic valve opening, and 608 indicates a vibration signal for the pulmonic valve opening. The data represented by the signals in FIG. 6 provide the necessary cardiac time intervals required for subsequent analysis as set forth below.

As noted above, in the context of the present disclosure, the term “cardiac time interval” refers to a time when an event occurs relative to an electrocardiogram. The time reference or registration comes from annotation of the simultaneously recorded ECG signal's Q or R wave. For instance, isovolumic contraction time (IVCT or the Mitral Valve Closure to Aortic Valve Opening period), isovolumic relaxation times (IVRT or Aortic Valve Closer to Mitral Valve Opening period), pre-ejection period (PEP or Q to Aortic Opening period), electomechanical delay (EMD) or Electromechanical activation time (EMAT) or Q to Mitral Closing period), left ventricular ejection time (LVET or Aortic valve opening to Aortic Valve Closure period), S1S2 or M1A2 (or closure of Mitral valve to Closure of Aortic valve), S2S1 or A2M1 (or closure of aortic valve to closure of the mitral valve of the next heart cycle), along with similar time intervals for the tricuspid and pulmonary valve, are examples of such time features.

In one implementation, a method of measuring a parameter indicative of left ventricular ejection fraction, LVEF, may comprise calculating LVEF from the linear equation formula LVEF=A1+A2*(PEP/LVET), where A1 and A2 denote predetermined numerical constants, PEP denotes the pre-ejection period, and LVET denotes the left ventricular ejection time. The numerical values of A1 and A2 in the formula above may be set, by way of example, according to C L Garrard, Jr., et. al., “The relationship of alterations in systolic time intervals to ejection fraction in patients with cardiac disease”, Circulation, September 1970; 42:455-462 (A1=0.84 and A2=0.64), according to L V Capan et al., “Measurements of ejection fraction by bioimpedance method”, Crit Med 1987; 15:402 (A1=1.125 and A2=1.25), or according to empirical data such as illustrated in FIG. 7 (A1=1.21, A2=2.17). Specifically, FIG. 7 illustrates Ejection Fraction measurements derived from PEP/LVET.

Even though the numerical values of A1 and A2 may differ in various embodiments, the quotient PEP/LVET may used in computation of LVEF. Since it is often not necessary to measure absolute values of LVEF, the exact values of A1 and A2 are not critical or outcome determinative; in that regard, it may be possible to use the quotient PEP/LVET as a replacement or proxy for LVEF in some applications. FIG. 7 illustrates regression results of measurements from patients by comparing hemodynamic measurements to results from an echocardiogram and to calculate accuracy of one embodiment. In another embodiment, it may be useful to use a linear regression model equation having the form Y(n)=b+Σwᵢ*xᵢ(n)+ε(n) (where Y(n) is the hemodynamic parameter, xᵢ(n) is the i^{th} cardiac time interval feature from the n^{th} context frame, wᵢ denotes a weight, b denotes a bias parameter, and ε(n) denotes independent and identically distributed zero mean observation noise), or a non-linear regression model equation having the form Y(n)=f(x(n))+β(n)=αᵢ*k(x(n), x(j))+ε(n)(where Y(n) is the hemodynamic parameter, xᵢ(n) is the i^{th} cardiac time interval feature from the n^{th} context frame, αⱼ denotes a weight, k denotes a positive definite kernel, and ε(n) denotes independent and identically distributed zero mean observation noise).

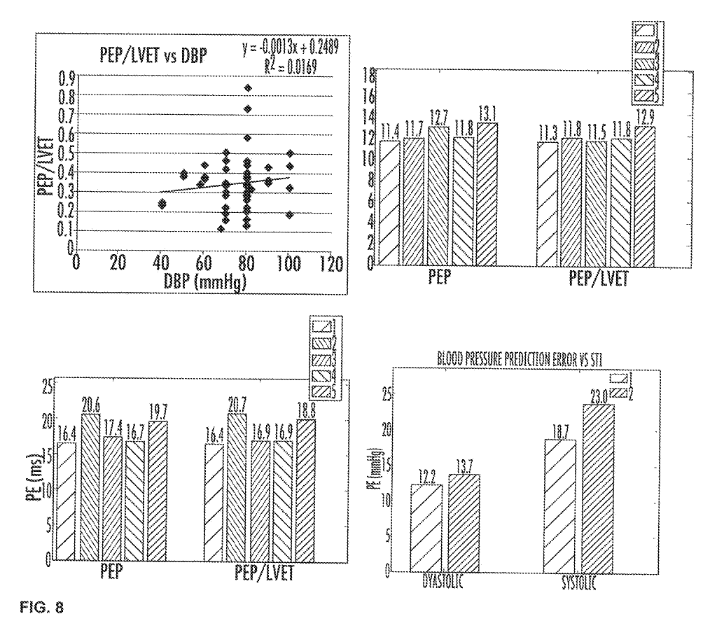

Additionally or alternatively, a method of measuring a parameter indicative of systolic blood pressure (SBP) and diastolic blood pressure (DBP) may generally comprise using the formula X=A1+A2*(S1S2/S2S1), where X=SBP or DBP, and where A1 and A2 again denote predetermined numerical constants, and S1S2 and S2S1 may be cardiac time intervals or a ratio of cardiac intervals. The numerical values of A1 and A2 in the formula above may be set, by way of example, according to empirical data such as illustrated in FIG. 8 (A1=−191.46, A2=769.23). Again, it is often not necessary to measure absolute values, and so it may be possible to use the quotient as a replacement or proxy for SBP or DBP in some applications, in connection with using linear, or linear regression or non-linear regression to derive the measurement. FIG. 8 illustrates regression results of measurements from patients by comparing measurement results to echocardiogram signals. Specifically, FIG. 8 illustrates blood pressure measurements derived from cardiac time intervals PEP/LVET; in some implementations, such measurements may be derived from cardiac time intervals S1S2 and S2S1 or other cardiac time intervals.

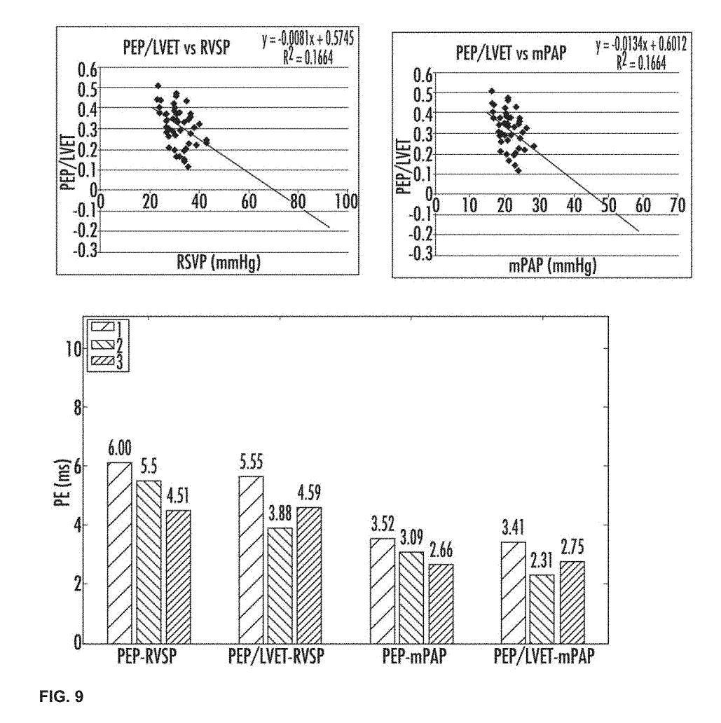

FIG. 9 illustrates mean pulmonary blood pressure measurement and right ventricular systolic pressure measurement from cardiac time intervals, such as PEP/LVET. In one example, a method of measuring a parameter indicative of mean pulmonary blood pressure (mPAP) may generally comprise calculating mPAP using the formula mPAP=A1+A2 (cardiac time interval), where A1 and A2 again denote predetermined numerical constants. Empirical data related to A1 and A2 are illustrated in FIG. 9, where A1=−70.92, A2=123.45. As with embodiments set forth above, it may be possible to use the quotient EMAT (electromechanical activation time) or other cardiac time intervals such as PEP/LVET in lieu of systolic PAP for some applications. It will be appreciated that a method of measuring a parameter indicative of right ventricular systolic pressure (RVSP) may generally comprise using the formula RVSP=A1+A2*(cardiac time interval), where A1 and A2 denote predetermined numerical constants. In some implementations, A1=−44.86 and A2=74.62, though other values may be used. As noted above, for certain computations, cardiac time intervals may be used instead of RVSP. In both instances, FIG. 9 illustrates regression results of measurements from patients by comparing hemodynamic measurements to results from an echocardiogram.

Additionally or alternatively, a method of measuring a parameter indicative of systolic dysfunction and diastolic dysfunction may generally comprise using the formula X=A1+A2* ((IVCT+IVRT)/LVET), where A1 and A2 again denote predetermined numerical constants. It may employ a similar formula using the different cardiac time intervals having the general form: A1+A2*(cardiac time intervals), or the linear regression form, Y(n)=b+Σwᵢ*xᵢ(n)+ε(n), or a non-linear regression model equation having the form Y(n)=f(x(n))+β(n)=Σαⱼ*k(x(n), x(j))+ε(n) as noted above.

Finally, it is noted that a method of measuring a parameter indicative of left and right ventricular ejection fraction, pulmonary artery systolic and diastolic blood pressure, systolic blood pressure and diastolic blood pressure, ventricular diastolic pressure, stroke volume, cardiac output, vascular resistance, global longitudinal strain, fluid status, E/E′, pulmonary capillary wedge pressure, left ventricle end diastolic volume and dP/dTmax may employ a similar formula using the different cardiac time intervals having the general form: A1+A2*(cardiac time intervals), where A1 and A2 denote predetermined numerical constants, or the form, Y(n)=b+Σwᵢ*xᵢ(n)+ε(n), or a non-linear regression model equation having the form Y(n)=f(x(n))+β(n)=Σαⱼ* k(x(n), x(j))+ε(n) as set forth in detail above.

According to yet another embodiment, a system providing hemodynamic parameter measurements may generally comprise a software algorithm for detecting opening and closing of the aortic valve, for instance, or some other event in a cardiac cycle based upon a suitable cardiac time interval. In that regard, such an algorithm may be implemented as source separation or source detection software to process data captured by transducer array 102, or example, at a particular cardiac time interval relative to an event monitored by an EKG. In the foregoing manner, noise, vibration, or another detectable signal associated with a particular event may be isolated as a function of, or facilitated by, a specific cardiac time interval synchronized to an EKG signal. Accordingly, various types of signals may be isolated or extracted from a composite signal for analysis, further processing, monitoring, storing in a patient-specific or population-specific database, or for a combination of these and other uses.

In some systems, a memory or digital data storage component or apparatus may be provided for storing values (for instance, of A1 and A2), raw sensor data, intermediate processing results, and other application-specific data or instructions; as noted above with reference to FIGS. 1 and 2, such memory or data storage may be implemented at wearable component 103, at vitals module 104, or both, depending upon their respective operational characteristics and overall system architecture. In any event, collected and stored values and other associated data may be transmitted or uploaded, for example, to an external receiver (such as vitals module 104 or remote processing component 105) on a regular basis, for example, or on-demand responsive to user input. In some instances, vitals module 104 may be implemented as a monitoring system resident at a patient's home or in a hospital room, and may receive data from wearable component 103 at predetermined intervals. Additionally or alternatively, remote processing unit 105 may be implemented as a remote monitoring or control system located remotely from a patient's location (for instance, in a server room at the patient's hospital or a server farm accessible via a network connection from the patient's home), and may receive data from vitals module 104 at predetermined intervals or on-demand responsive to user input. In some applications, regular and automatic (e.g., on a weekly or daily basis) data transmission may provide efficiency and ensure that attending physicians get important information in a timely fashion, even when remote from patients' locations. This may be especially useful in certain treatments or with respect to monitoring of certain conditions, such as may be required or desirable during administration of certain drugs or with respect to titration of diuretics.

According to yet another aspect, a system may receive as input a reference hemodynamic parameter measurement from a calibrated medical monitoring device (such as a blood pressure cuff, echocardiogram, or cardio-catheter, though other devices may be used) for calibration purposes. Using such a reference signal for calibration, a system and method as set forth above may provide calibrated hemodynamic measurements using cardiac time intervals as described.

As set forth above, it may be useful to implement a system and method of calculating hemodynamic measurements from heart valve signals that employ separated signal sources representative of individual heart vibration events that have been isolated or extracted from a composite signal representative of a plurality of vibration objects captured via multiple transducers. In one embodiment, such a system may be capable of identifying individual cardio-pulmonary events from the various vibration signals collected by an array of one or more sensors. A source separation algorithm, operative to isolate signals representative of individual events, may have utility with arbitrarily arranged multiple sensors; a system and method as set forth above may be capable of working with two or more transducers (though it may be possible to employ only a single sophisticated sensor for some applications) and handling multiple sources in a reverberant and noisy environment.

Capable of separating more sources than number of microphones (Underdetermined use-case). It handles spatial aliasing and the algorithms are able to separate signals in media that is non-linear, has time varying propagation, and heavily reverberant. It does not explicitly require sensor location information and can be arranged in any two or three dimensions' form.

A system and method of hemodynamic parameter measurement using source separation have been described which employ pulmonary and aortic signals, and possibly tricuspid and mitral auscultation locations, thus identifying occurrence of individual valve events and registering or synchronizing such events with respect to an electrocardiogram signal. Accordingly, the disclosed system and method are capable of measuring time intervals of cardio-pulmonary events relative to others.

It will be appreciated that the disclosed system and method may allow both short-term and long-term discrimination between signals. In this context, the phrase “short-term” generally pertains to tracking individual streams (i.e., isolating a respective particular source for a respective component of a composite signal) when they are captured simultaneously as part of a composite signal, whereas the phrase “long-term” generally pertains to tracking individual streams across multiple heart beats, for example, or tracking valve signals as they transition in and out during each cardiac cycle.

In another configuration, a non-invasive estimation of hemodynamic parameters based on source identification and separation may be implemented as an adaptation of U.S. Pat. No. 6,227,4761 filed on Feb. 10, 2010, entitled “Method and System of an Acoustic Scene Analyzer for Body Sounds,” the entire contents of which are hereby incorporated by reference.

Vitals module 103 or other components of system 100 may also be deployed in a clinical setting and configured to perform non-invasive estimation of hemodynamic parameters in combination with cardio-acoustic classification, screening, diagnosis and monitoring of cardiovascular condition as disclosed in pending U.S. patent application Ser. No. 13/370,514 filed on Feb. 10, 2010, entitled “Method and System of a for Cardio-acoustic Classification system, for Screening, Diagnosis and Monitoring of Cardiovascular Conditions”, Docket PAT002, the entire contents of which are hereby incorporated by reference.

Vitals module 103 or other components of system 100 may also be deployed in a clinical setting and configured to perform non-invasive estimation of hemodynamic parameters as disclosed in pending U.S. patent application Ser. No. 14/310,820 filed on Jun. 20, 2014, entitled “Method and System of Non-Invasive Estimation of Pulmonary Arterial Pressure and Ejection Fraction”, Docket PAT005, the entire contents of which are hereby incorporated by reference.

Several features and aspects of a system and method have been illustrated and described in detail with reference to particular embodiments by way of example only, and not by way of limitation. Those of skill in the art will appreciate that alternative implementations and various modifications to the disclosed embodiments are within the scope and contemplation of the present disclosure. Therefore, it is intended that the invention be considered as limited only by the scope of the appended claims.

What is claimed is:

1. A method of measuring hemodynamic parameters; said method comprising:
receiving a composite signal representative of emanations from a plurality of sources associated with a patient's cardio-pulmonary system;
isolating an individual signal as a separate component of the composite signal, the individual signal representative of an emanation from one of the plurality of sources; and
deriving hemodynamic parameters responsive to said isolating.

2. The method of claim 1 further comprising providing an output related to the individual signal and the hemodynamic parameters.

3. The method of claim 1 wherein said receiving comprises employing an array of transducers, and wherein each respective transducer in the array is disposed in a respective location on a patient.

4. The method of claim 1 wherein said isolating comprises measuring a cardiac time interval for the individual signal with respect to an electrocardiogram signal.

5. The method of claim 4 wherein said deriving comprises utilizing an equation having the form
Y=A1+A2*(A3)
where Y is the hemodynamic parameter, A1 and A2 denote predetermined numerical constants, and A3 denotes a cardiac time interval or a ratio of two cardiac time intervals.

6. The method of claim 5 wherein the predetermined numerical constants A1 and A2 are based upon empirical data.

7. The method of claim 5 wherein said deriving comprises utilizing a linear regression model equation having the form
Y(n)=b+Σwᵢ*xᵢ(n)+ε(n)
where Y(n) is the hemodynamic parameter, xᵢ(n) is the i^{th} cardiac time interval feature from the n^{th} context frame, wᵢ denotes a weight, b denotes a bias parameter, and ε(n) denotes independent and identically distributed zero mean observation noise.

8. The method of claim 5 wherein said deriving comprises utilizing a non-linear regression model equation having the form
Y(n)=f(x(n))+β(n)=Σαⱼ*k(x(n), x(j))+ε(n)
where Y(n) is the hemodynamic parameter, xᵢ(n) is the i^{th} cardiac time interval feature from the n^{th} context frame, αⱼ denotes a weight, k denotes a positive definite kernel, and ε(n) denotes independent and identically distributed zero mean observation noise.

9. A method of determining hemodynamics parameters; said method comprising:
sensing contractility changes within a heart by identifying a cardiac time interval;
sensing an electrocardiogram (EKG) signal representative of heart function; and
deriving the hemodynamic measurements using the sensed contractility changes and the EKG signal.

10. The method of claim 9 wherein said sensing contractility changes comprises measuring systolic time intervals of the heart and diastolic time intervals during a period from a start of a Q-wave of the EKG signal to a time when a change is sensed in the cardiac contractility.

11. The method of claim 10 wherein said deriving the hemodynamic measurements comprises utilizing contractility estimates to determine a ventricular systolic pressure and a ventricular diastolic pressure.

12. The method of claim 11 further comprising multiplying the diastolic time interval by the ventricular diastolic pressure, multiplying the systolic time interval by the ventricular systolic pressure, and adding the products to obtain a mean pulmonary artery pressure.

13. The method of claim 10 wherein said deriving the hemodynamic measurements comprises utilizing contractility estimates to determine a systolic blood pressure and a diastolic blood pressure.

14. The method of claim 10 wherein said deriving the hemodynamic measurements comprises utilizing contractility estimates to determine an ejection fraction.

15. The method of claim 10 wherein said deriving the hemodynamic measurements comprises utilizing contractility estimates to determine a stroke volume and wherein a product of the stroke volume and a heart rate determines cardiac output.

16. The method of claim 10 wherein said deriving the hemodynamic measurements comprises utilizing contractility estimates to determine a vascular resistance.

17. The method of claim 10 wherein said deriving the hemodynamic measurements comprises utilizing contractility estimates to determine a global longitudinal strain.

18. The method of claim 10 wherein said deriving the hemodynamic measurements comprises utilizing contractility estimates to determine a fluid status of a person.

19. The method of claim 10 wherein said deriving the hemodynamic measurements comprises utilizing contractility estimates to determine systolic dysfunction and diastolic dysfunction.

20. A system or monitoring cardiac parameters; said system comprising:
a non-invasive cardiac parameter measuring unit for non-invasively measuring a plurality of predetermined non-invasive cardiac parameters from a subject;
a conversion unit connected to said non-invasive cardiac parameter measuring unit for converting the non-invasive cardiac parameters into a plurality of invasive cardiac analogues based upon a set of predetermined conversion equations and;
a display unit connected to said conversion unit for displaying a vector indicative of a hemodynamic state.
